(19) United States
(12) Patent Application Publication
(10) Pub. No.: US 2006/0228795 A1
(43) Pub. Date: Oct. 12, 2006

(54) Apparatus for enhancing proliferation of cells in a small-scale cell culturing container

(76) Inventor: Clayton R. Parker, Safety Harbor, FL (US)

Correspondence Address: LADAS & PARRY LLP, 224 SOUTH MICHIGAN AVENUE, SUITE 1600, CHICAGO, IL, 60604, US

(21) Appl. No.: 11/169,613

(22) Filed: Jun. 29, 2005

Related U.S. Application Data
(60) Provisional application No. 60/584,508, filed on Jun. 30, 2004.

(51) Int. Cl. C12N 5/06; C12M 3/00

(52) U.S. Cl. 435/325; 435/289.1

(57) ABSTRACT

An interchangeable sleeve for enhancing proliferation of cells in a small-scale culturing container is disclosed with the interchangeable sleeve having a substantially cylindrical and substantially rigid electrical conductive material wound in a cylindrical shape and capable of being connected to a pulsating time varying electromagnetic current to create a time varying electromagnetic force of from approximately 0.05 gauss to 0.5 gauss within the cylindrical portion of the sleeve.

### BACKGROUND OF THE INVENTION

1. Field of the Invention

The present invention relates generally to a device for enhancing proliferation of cell cultures in a small-scale culturing container such as a petri dish. Specifically, this invention relates to an interchangeable sleeve that encompasses a small-scale culturing container with the sleeve supplying a time varying electromagnetic force to the small-scale culturing container in order to increase cell growth and proliferation within the small-scale culturing container. More specifically, the present invention relates to an interchangeable sleeve that can encompass a small-scale culturing container and supply a time varying electromagnetic force of from 0.05 gauss to 0.5 gauss to the bioreactor chamber to substantially increase cell growth and proliferation within the small-scale culturing container.

The preferred embodiment is an interchangeable sleeve for enhancing proliferation of cells in a small-scale culturing container, with the sleeve comprising: a substantially cylindrical and substantially rigid electrical conductive material wound in a cylindrical shape and capable of being connected to a pulsating time varying electromagnetic current to create a time varying electromagnetic force of from approximately 0.05 gauss to 0.5 gauss within the cylindrical portion of the sleeve; and means for applying a pulsating time varying electromagnetic current to the electrical conductive material to create the time varying electromagnetic force of from approximately 0.05 gauss to 0.5 gauss within the cylindrical portion of the sleeve.

2. Description of the Prior Art

Wolf and Goodwin, in U.S. Pat. No. 6,673,597, patented the use of a time varying electromagnetic force to develop and proliferate cell cultures. Commercial utilization of this technology has provided two approaches to bioreactor design. The first approach is the use of baffles or plates within the bioreactor culture chamber with a time varying electromagnetic current applied across the plates to induce a time varying electromagnetic force within the culture chamber. The second approach is the use of a coil wrapped around a rotating bioreactor chamber and affixed thereto with a time varying electromagnetic current applied to the coil to create a time varying electromagnetic force within the culture chamber.

The problem with the prior art designs for application of a time varying electromagnetic force (TVEMF) to a bioreactor chamber is that the coil used to induce the TVEMF or the plates within the bioreactor are part of the bioreactor chamber. Since the goal of proliferation of cell cultures is in many instances the utilization of the cell cultures for reintroduction into the human body for tissue regeneration or treatment of human maladies, the bioreactor chamber must meet the rigid standards of the Food and Drug Administration. Consequently, rather than comply with the rigid requirements the Food and Drug Administration for cleaning the bioreactor chamber to guarantee there is no contamination of the cell culture within the chamber, it would be highly desirable to have a disposable culture chamber. With a disposable chamber, the culture chamber could be manufactured pursuant to the rigid requirements the Food and Drug Administration and packaged in a sterile environment and container thereby enabling it to be used by the medical or research professional much the same as other disposable medical devices are used. However, if the TVEMF inducing device is incorporated into the disposable culture chamber, it significantly complicates the manufacture and sterilization process, and it requires that the TVEMF inducing device be disposed of along with the discarding of a used bioreactor chamber thereby significantly adding to the cost of the equipment and culturing process.

It would be highly desirable to provide disposable small-scale culturing containers without the TVEMF inducing device being an integral part thereof.

The present invention overcomes problems associated with prior art bioreactor designs by allowing disposable small-scale culturing containers to be provided without the need to dispose of the small-scale culturing containers TVEMF inducing device.

### SUMMARY OF THE INVENTION

The present invention relates to an interchangeable sleeve for enhancing proliferation of cells in small-scale culturing containers. The interchangeable sleeve is used to encompass small-scale culturing containers, such as a petri dish, and is designed to impart a time varying electromagnetic force of from 0.05 gauss to 0.5 gauss to the small-scale culturing containers.

The interchangeable sleeve of this invention is an independent time varying electromagnetic wave generating coil that can be used with small-scale culturing containers to significantly increase cell growth and proliferation in the small-scale culturing containers.

It is an object of this invention to provide an interchangeable sleeve for use with small-scale culturing containers.

It is a further object of this invention to provide an interchangeable sleeve for use with small-scale culturing containers wherein the interchangeable sleeve imparts a time varying electromagnetic force (square wave, Fourier curve) to the small-scale culturing containers.

Other aspects, features and advantages of the present invention will be apparent from the following description of the presently preferred embodiments of the invention given for the purpose of disclosure.

### BRIEF DESCRIPTION OF THE DRAWINGS

In the drawings,

FIG. 1 shows the assembly of the apparatus in conjunction with a petri dish.

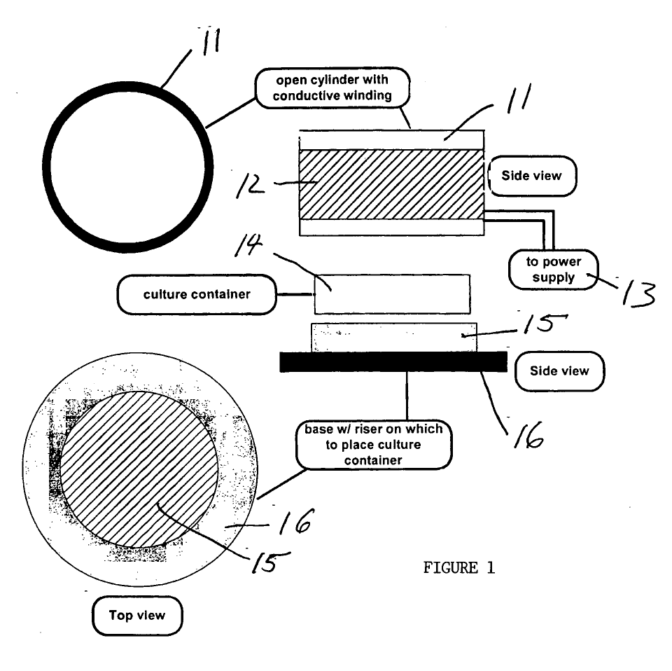

### DETAILED DESCRIPTION OF THE INVENTION

In the drawings, a cylinder 11 has a wire coil 12 wound around it. The wire coil has approximately 10 windings per inch. The coil is connected to a power supply 13 that provides a square wave (Fourier curve) time varying electrical current to the wire coil 12 thereby inducing a square wave (Fourier curve) time varying electromagnetic force of from 0.05 gauss to 0.5 gauss to the interior of the coil. A small-scale culturing container, such as a petri dish 14, is placed on a support device 15 that sits on a stand 16. The support device 15 and stand 16 are used in combination to elevate the petri dish 14 so it is well within the time varying electromagnetic force generated by the wire coil 12.

In application collected peripheral blood cells PBCs (0.75×10⁶ cells/ml) obtained from donors are suspended in Iscove's modified Dulbecco's medium (IMDM) (GIBCO, Grand Island, N.Y.) supplemented with 5% human albumin (HA) or 20% human plasma, 100 ng/ml recombinant human G-CSF (Amgen Inc., Thousand Oaks, Calif.), and 100 ng/ml recombinant human stem cell factor (SCF) (Amgen). D-Penicillamine [D(−)-2-Amino-3-mercapto-3-methylbutanoic acid] (Sigma-Aldrich) a copper chelation agent, is dissolved in DMSO. 10 ppm of the D-Penicillamine is introduced into the cell mixture. One sample of the culture mix is placed into the petri dish described herein. A time varying electromagnetic force of approximately 0.5 gauss was created in the cylinder that was over the petri dish. A second sample was placed in a petri dish without any time varying electromagnetic force applied thereto.

After the seventh day of expansion, the cells were washed with PBS and analyzed by conventional counting techniques, for example by using a Coulter counter. The sample exposed to the time varying electromagnetic force had more than twice the growth or expansion of the sample that was not exposed to the time varying electromagnetic force.

Having fully described this new and unique invention, I claim:

1. An interchangeable sleeve for enhancing proliferation of cells in a small-scale culturing container, said sleeve comprising:
a. a substantially cylindrical and substantially rigid electrical conductive material wound in a cylindrical shape and capable of being connected to a pulsating time varying electromagnetic current to create a time varying electromagnetic force of from approximately 0.05 gauss to 0.5 gauss within the cylindrical portion of the sleeve; and
b. means for applying a pulsating time varying electromagnetic current to the electrical conductive material to create the time varying electromagnetic force of from approximately 0.05 gauss to 0.5 gauss within the cylindrical portion of the sleeve.

2. An interchangeable sleeve as in claim 1 wherein the electrical conductive material is electrical conductive wire.

3. An interchangeable sleeve as in claim 1 wherein the time varying electromagnetic force is a square wave.

4. An interchangeable sleeve as in claim 2 wherein the electrical conductive wire is wound in the cylindrical shape at approximately ten windings per inch.

5. An interchangeable sleeve as in claim 1 wherein the substantially cylindrical and substantially rigid electrical conductive material wound in a cylindrical shape is electrical conductive wire wound about an electromagnetically permeable polymer with a substantially cylindrical shape.

6. An interchangeabel sleeve as in claim 5 wherein the electrical conductive wire is wound in the cylindrical shape at approximately ten windings per inch.

7. An inter changeable sleeve as in claim 5 wherein the electrical conductive wire is a ferromagnetic metal.
